(19) United States
(12) Patent Application Publication
(10) Pub. No.: US 2009/0011017 A1
(43) Pub. Date: Jan. 8, 2009

(54) POLYMERS BASED ON WATER-SOLUBLE MONOOLEFINIC COMPRISING COLLOIDAL SILICA AND THEIR USE AS MATRIX POLYMERS FOR SOLID DOSAGE FORMS

(75) Inventors: Murat Mertoglu, Ludwigshafen (DE); Karl Kolter, Limburgerhof (DE); Klemens Mathauer, Taipei (TW); Gerhard Rossler, Neuhofen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/164,246

(22) Filed: Jun. 30, 2008

(30) Foreign Application Priority Data
Jul. 5, 2007 (EP) 07111849.1

(51) Int. Cl. A61K 9/22; C08F 18/00

(52) U.S. Cl. 424/468; 526/194

(57) ABSTRACT

Copolymers obtained by free-radical polymerization of a mixture of
a) 80 to 99% by weight of a monoolefinic monomer selected fronm the group consisting of acrylic monomers, methacrylic monomers and N-vinyllactam monomers (monomers a)),
b) 1 to 20% by weight of a monoolefinic silane monomer (monomer b)),
   with the proviso that the total of components a) and b) equals 100% by weight,

The present invention relates to novel polymers based on monoolefinic monomers and colloidal silica, a process for manufacturing such polymers and their use as matrix polymers in pharmaceutical or cosmetic preparations.

Oral pharmaceutical dosage forms with delayed release or sustained release of active ingredients are becoming increasingly important. Besides coated dosage forms where control of the release is achieved by coating cores containing the active ingredient with a film which is insoluble in water but semi-permeable or contains pores through which the active ingredient diffuses, it is also possible to achieve control of the release and prolongation of releases by embedding the active ingredient in a matrix which acts as a release controlling matrix. Such matrix delayed release offers the advantage of simple and low-cost production and, in a addition, enhanced safety due to avoidance of dose dumping effect.

Ancillary substances usually applied for this are natural or semi-synthetic polymers such as hydroxypropylcellulose, hydroxypropylmethylcellulose, alginates, xanthan gums and the like.

DE-A 2528068 relates to cross-linked water-soluble hydrophilic gels consisting, inter alia, of N-Vinylpyrrolidone-containing polymers with various water-insoluble monomers such as alkyl acrylates and alkyl methacrylates and bi-functional olefinic macromers and their use as matrix polymers in pharmaceutical dosage forms.

JP 07228639 A relates to thermosensitive crosslinked hydrogels obtained from acrylic and/or vinyllactame monomers which are crosslinked by either copolymerizing poly-functional monomers or polyvalent metal ions and which comprise inorganic polymer systems such as silicone or phosphazene systems.

U.S. Pat. No. 6,436,440 describes uncrosslinked matrix polymers for dosage forms with delayed release of the active ingredient which polymers are consisting of N-Vinyllactams and, hydrophobic monomers. However, tablets obtained by compression of such polymers show disadvantages with regard to tablet hardness and friability.

It is an object of the present invention to find polymers which are suitable as matrix polymers for pharmaceutical or cosmetic preparations with sustained release of the active ingredient and which show enhanced properties with regard to release control, tablet hardness and friability.

We have found that this object is achieved by copolymers obtained by radical polymerization of at least one water-soluble monoolefinic monomer selected from the group consisting of basic N-Vinyl monomers, acrylic monomers and methacrylic monomers (Monomers a) and a monoolefinic silane monomer (Monomer b) in the presence of colloidal silica, with the proviso that the total amount of monomers a) and b) equals 100% b.w.

Preferably the polymers are obtained from 80 to 99 % b.w. of monomer a) and 1 to 20% b.w. of monomer b), more preferably 85 to 95% b.w. of monomer a) and 5 to 15% b.w. of monomer b), particularly 87 to 90% b.w. monomer a) and 10 to 13% b.w. of monomer b).

Colloidal silica is employed in amounts of 0,1 to 40% b.w. , preferably 0,2to 20% b.w., based on the total amount of monomers a) and b).

Monomers a) useful for the present invention are selected from the group consisting of basic N-vinyl such as N-vinyllactames, N-vinylimidazole or N-vinyl carboxamides. Suitable N-vinyl lactames are N-vinyl pyrrolidon, N-vinyl caprolactam, N-vinyl piperidone, N-vinylpyridine. Preferred N-vinyl lactames are N-vinyl pyrrolidone and N-vinyl caprolactame or mixtures of thereof.

Further monomers a) are selected from the group consisting of acrylic and methacrylic monomers such as methacrylamide, dimethyl methacrylamide dimethyl acrylamiden-isopropylmethacrylamide, n-isopropylacrylamide, poly(ethyleneglycole) methylether methacrylate

Monomers b) useful for the present inventions are acryloyloxy-, methacryloyloxy- or vinyloxy- alkyl trialkoxy silanes of the general formula R¹—(CH₂)ₙ—Si(R²)₂R³, where in R1 means acryloyloxy-, methacryloyloxy - or vinyloxy-, n=1 to 10, R² is —O(—CH₂—)ₘCH₃ with m=0 to 4 and R³ is either ═R² or —H, —CH₃, —CH₂CH₃.

A particularly preferred silane monomer is (3-methacryloyloxy)-propyl trimethoxy sitane.

According to the present invention the monomers a) and the silane monomer are co-polymerised in the presence of colloidal amorphous silica. Preferably the colloidal silica shows medium particle sizes of 5 nm to 200 nm, more preferably medium particle sizes of 5 to 75 nm. The colloidal silica can be added to the polymerization mixture as a solid powder or in the form of an aqueous dispersion. Such colloidal silica is commercially available, for instance under the brand name Levasil®.

The preparation of the novel polymers takes place by free-radical polymerization, preferably solution polymerization, in water or in mixed nonaqueous/aqueous solvents.

Suitable nonaqueous organic solvents which could be used for water mixtures are, for example, alcohols such as methanol, ethanol, n-propanol and isopropanol, and glycols such as ethylene glycol and glycerol.

The polymerization is preferably carried out at temperatures from 50 to 100° C.

Free-radical initiators are employed to initiate the polymerization. The amounts of initiator or initiator mixtures used, based on monomer employed, are between 0.01 and 10% by weight, preferably between 0.3 and 5% by weight.

Depending on the nature of the solvent used, both organic and inorganic peroxides are suitable. Examples of peroxide initiators are dibenzoyl peroxide, diacetyl peroxide, succinyl peroxide, tert-butyl perpivalate, tert-butyl 2-ethylhexanoate, tert-butyl permaleate, bis-(tert-butylperoxy)cyclohexane, tert-butylperoxy isopropyl carbonate, tert-butyl peracetate, 2,2-bis(tert-butylperoxy)butane, dicumyl peroxide, di-tert-amyl peroxide, di-tert-butyl peroxide, p-menthane hydroperoxide, pinane hydroperoxide, cumene hydroperoxide, tert-butyl hydroperoxide, hydrogen peroxide sodium persulfate and mixtures of said initiators. Said initiators can also be used in combination with redox components such as ascorbic acid. Particularly suitable peroxide initiators are tert-butyl perneodecanoate, tert-butyl perpivalate or tert-butyl 2-ethylhexanoate.

Preferably, the free-radical initiators are azo initiators. Suitable azo initiators are 2,2′-Azobis[2-(2-imidazolin-2-yl)propane]dihydrochloride, 2,2′-Azobis[2-(2-imidazolin-2-yl)propaneldisulfate dihydrate, 2,2′-Azobis(2-methylpropionamidine)dihydrochloride, 2,2′-Azobis[N-(2-carboxyethyl)-2-methylpropionamidine]hydrate, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis(1-imino-1-pyrrolidino-2-ethylpropane)dihydrochloride, 2,2′-Azobis{2-methyl-N-[1,1-bis(hydroxymethyl)-2-hydroxyethl]propionamide, 2,2′-Azobis[2-methyl-N-(2-hydroxyethyl)propionamide].

The monomer or a monomer mixture or the emulsion of monomer(s) may be introduced together with the initiator, which is generally present in solution, into a stirred reactor at the polymerization temperature (batch process) or metered continuously or in a plurality of consecutive stages into the polymerization reactor (feed process). It is usual in the feed process for the reactor to be charged, before the start of the actual polymerization, besides the solvent (in order to make stirring of the reactor possible) also with partial quantities, rarely the total quantity intended for the polymerization, of the starting materials such as emulsifiers, protective colloids, monomers, regulators etc. or partial quantities of the feeds (generally monomer feed or emulsion feed and initiator feed). Preferably, the polymers according to the present invention are obtained by a feed process.

The polymer dispersions or solutions can be converted into powder form or into granules by various drying processes such as, for example, spray drying, fluidized spray drying, drum drying or freeze drying.

The polymers according to the present invention are hydrogels with a good water-absorption and water-retention capacity. Due to these properties the copolymers will swell in contact with aqueous media such as gastro-intestinal fluids and thus cause sustained release of the active ingredient. Sustained release means that the amount of active ingredient released after 3 and up to 24 hours is greater than 80% b.w. of the total amount of active ingredient.

The hydrogels according to the present invention do not show a critical solution temperature or temperature depending phase transitions.

The novel copolymers are useful for all pharmaceutical or cosmetic dosage forms where prolonged release of the active is desired. Pharmaceutical active ingredients according to the present invention shall also comprise vitamins, nutraceuticals such as β-carotin, lycopin or other carotenoids or dietary supplements.

Examples of pharmaceutical active ingredients which may be mentioned here are benzodiazepines, antihypertensives, vitamins, cytostatics—especially Taxol, anesthetics, neuroleptics, antidepressants, agents having antiviral activity, antibiotics, antimycotics, fungicides, chemotherapeutics, urologicals, platelet aggregation inhibitors, sulfonamides, spasmolytics, hormones, immunoglobulins, sera, thyroid therapeutics, psychoactive drugs, antiparkinson agents and other antihyperkinetics, ophthalmologicals, neuropathy products, calcium metabolism regulators, muscle relaxants, anesthetics, lipid-lowering agents, hepatotherapeutics, coronary agents, cardiac agents, immunotherapeutics, regulatory peptides and their inhibitors, hypnotics, sedatives, gynecologicals, gout remedies, fibrinolytics, enzyme products and transport proteins, enzyme inhibitors, emetics, blood flow stimulators, diuretics, diagnostic aids, corticoids, cholinergics, biliary therapeutics, anti asthmatics, bronchodilators, beta-receptor blockers, calcium antagonists, ACE inhibitors, arteriosclerosis remedies, antiinflammatory drugs, anticoagulants, antihypertensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, slimming agents.

In matrix dosage forms the novel copolymer is usually comprised in amounts of 0.5 to 90, preferably 20 to 80% b.w., based on the total weight of the dosage form.

In addition, the dosage forms may comprise customary pharmaceutically acceptable excipients and auxiliaries such as bulking agents, binders, glidants, lubricants, colouring agents, surfactants, salts or dispersing aids. These may be inter alia:

Bulking agents and binders such as, for example, lactose, calcium phosphates, cellulose and cellulose derivatives, starch and starch derivatives, polyvinylpyrrolidone, polyvinylalcohol, partially hydrolyzed polyvinylacetate, sugar alcohols, sugras, waxes, fats Glidants and lubricants such as, for example highly disperse silica, Mg stearate or stearic acid.

The dosage forms may be coated with one or more coating layers such as for instance acid resistant coatings.

### EXAMPLES

### Polymer Preparation

The silane ester used in Ex. 1 to 3 was (3-methacryloyloxy)-propyl trimethoxy silane.

### Example 1

### Feed Material Allocation

| | | |
|---|---|---|
| | Initial charge | 250 g water |
| Feed 1 | water | 500 g |
| | N-vinylpyrrolidone | 87.5 g |
| | silane ester | 12.5 g |
| | Levasil ® 200 A*) | 1.88 g |
| Feed 2 | water | 50 g |
| | 2,2′-azobis(2-amidinopropane) dihydrochloride | 2 g |
| | (Wako ® V50) | |

*)commercially available aqueous dispersion of amorphous colloidal silica with a specific surface of 200 m²/g with a silica content of 40% b.w..

The polymerization took place in a stirred reactor with a volume of 2 l. The initial charge was flushed with nitrogen then heated to the polymerization temperature T (° C.) (internal temperature) of 80° C.

At 80° C. feeds 1 and 2 were started. Feed 1 was metered in over 4 hours, feed 2 over 4.5 hours. The mixture was then after-polymerized for a hours. The resulting polymer was a clear gel The gel was dried in a vacuum oven at 50 C°.

### Example 2

### Feed Material Allocation

| | | |
|---|---|---|
| | Initial charge | 350 g water |
| | 15.7 g N-vinyl pyrrolidone | |
| | 4.8 g N-vinyl caprolactame | |
| Feed 1 | water | 500 g |
| | N-vinylpyrrolidone | 76 g |
| | N-vinylcaprolactame | 26 g |
| | silane ester | 17.5 g |
| | Levasil 200 A | 5.2 g |
| Feed 2 | water | 70 g |
| | 2,2′-azobis(2-amidinopropane) dihydrochloride | 2.8 g |
| | (Wako V50) | |

The polymerization took place in a stirred reactor with a volume of 2 l. The initial charge was flushed with nitrogen and then heated to the polymerization temperature T (° C.) (internal temperature) of 80° C.

At 80° C. feeds 1 and 2 were started. Feed 1 was metered in over 4 hours, feed 2 over 4.5 hours. The mixture was then after-polymerized for a hours. The resulting polymer was a clear gel. The gel was dried in a vacuum oven at 50 C°.

### Example 3 (Comparative Example)

### Feed Material Allocation

| | | |
|---|---|---|
| | Initial charge | 250 g water |
| Feed 1 | water | 500 g |
| | N-vinylpyrrolidone | 87.5 g |
| | silane ester | 12.5 g |
| Feed 2 | water | 50 g |
| | 2,2′-azobis(2-amidinopropane) dihydrochloride | 2 g |
| | (Wako V50) | |

The polymerization took place in a stirred reactor with a volume of 2 l. The initial charge was flushed with nitrogen then heated to the polymerization temperature T (° C.) (internal temperature) of 80° C.

At 80° C. feeds 1 and 2 were started. Feed 1 was metered in over 4 hours, feed 2 over 4.5 hours. The mixture was then after-polymerized for a hours. The resulting polymer was a clear highly viscous solution. The solution was dried in a vacuum oven at 50 C°.

TABLE 1
% b.w. release of propanolol vs. time with different polymers

| Time (h) | *) | Eample 1 | Example 2 | Example 3 |
|---|---|---|---|---|
| 0 | 0 | 0 | 0 | * |
| 1 | 18 | 14 | 25 | |
| 2 | 27 | 22 | 36 | |
| 4 | 31 | 31 | 56 | |
| 8 | 47 | 49 | 89 | |
| 12 | 58 | 63 | 99 | |
| 16 | 67 | 77 | 99 | |
| 20 | 75 | 84 | — | |
| 24 | 81 | 90 | — | |

* in 2 hours complete release
*) Poly(Vinylpyrrolidone-co-stearylmethacrylat) (70:30). K-value 1 wt. % in ethanolic solution between 40-50.

### Determination of Swelling Properties

In order to determine the swelling properties of a polymer sample the Free Swell Capacity (FSC) and the Centrifuge Retention Capacity (CRC) were measured according to the following method.

0.2 g of a polymer sample in a tightly sealed tea bag was placed in distilled water for 30 min. Then the tea bag was left to drip free from water for 10 min. and weighed. FSC was calculated as amount of water absorbed per g polymer. Subsequently, the tea bag was placed in centrifuge for 3 min. at 1400 rpm and weighed. CRC was calculated as amount of water retained per g polymer.

| Polymer of ex. No. | FSC g/g | CRC g/g |
|---|---|---|
| 1 | 12.7 | 9.4 |
| 2 | 9.6 | 4.8 |

### Preparation of Tablets

| | |
|---|---|
| Propranolol Hydrochloride | 160 mg |
| Copolymer | 160 mg |
| Highly disperse silica | 3.4 mg |
| Magnesium stearate | 1.6 mg |

The components were sieved (0.8 mm mesh) and mixed for 10 min in a Turbula mixer. Tablets were pressed at 18 kN on an excenter press, 30 hubs/min, 10 mm punches.

TABLE 2
Mechanical comparison of the tablets prepared with polymers

| | Strength at break | Friability |
|---|---|---|
| Ex. 1 | 78 N | <0.1% |
| Ex. 2 | 68 N | <0.1% |
| Ex. 3 | <10 N | Tablets disintegrate |

The release properties were tested in a paddle apparatus: 2 hours in 0.08 n HCl, then 22 hours in phosphate buffer at pH 6.8

1-12. (canceled)

13. A copolymer obtained by free-radical polymerization of a mixture of
a) 80 to 99% by weight of a monoolefmic monomer (monomer (a)) selected from the group consisting of acrylic monomers, methacrylic monomers, and N-vinyllactam monomers;
b) 1 to 20% by weight of a monoolefinic silane monomer (monomer (b));
in the presence of colloidal amorphous silica, with the proviso that the total of monomer (a) and monomer (b) equals 100% by weight.

14. The copolymer of claim 13, wherein said copolymers are obtained from 85 to 95% by weight of monomer (a) and 5 to 15% by weight of monomer (b).

15. The copolymer of claim 13, wherein said N-vinyllactams are N-vinylpyrrolidone, N-vinylcaprolactam, or mixtures thereof.

16. The copolymer of claim 13, wherein said acrylic monomers and methacrylic monomers are selected from the group consisting of methacrylamide, dimethyl methacrylamide, dimethyl acrylamide, n-isopropylmethacrylamide, n-isopropylacrylamide, and poly(ethyleneglycol) methylether methacrylate.

17. The copolymer of claim 13, wherein monomer (b) is selected from the group consisting of acryloyloxy-, methacryloyloxy-, and vinyloxy- alkyl trialkoxy silanes of general formula R¹—(CH₂)ₙ—Si(R²)₂R³, wherein R¹ means acryloyloxy-, methacryloyloxy- or vinyloxy-; n is an integer from 1 to 10; R² is —O(—CH₂—)ₘCH₃, wherein m is an integer from 0 to 4; and R³ is either R² or —H, —CH₃, —CH₂CH₃,

18. The copolymer of claim 13, wherein monomer (b) is (3-methacryloyloxy)-propyl methoxy silane.

19. The copolymer of claim 13, wherein said colloidal amorphous silica is used in an amounts of from 0.1 to 40% by weight based on the total amount of monomer (a) and monomer (b).

20. The copolymer of claim 13, wherein said colloidal amorphous silica has a medium particle size of from 5 to 200 nm.

21. A solid pharmaceutical dosage form with sustained release of the active ingredient comprising as a matrix component 0.5 to 90% by weight of the copolymer of claim 13.

22. The solid dosage form of claim 21, wherein at least 80% by weight of said active ingredient is released after 3 to 24 hours following administration.

23. The solid dosage form of claim 21, further comprising customary ancillary substances selected from the group consisting of bulking agents, binders, glidants, lubricants, colouring agents, surfactants, salts, and dispersing aids.
